(19) United States
(12) Patent Application Publication
(10) Pub. No.: US 2007/0255297 A1
(43) Pub. Date: Nov. 1, 2007

(54) Apparatus for Cutting a Corneal Epithelial Cover

(76) Inventor: Antoine Sempe, Issy-Les-Moulineaux (FR)

Correspondence Address: YOUNG & THOMPSON, 745 SOUTH 23RD STREET, 2ND FLOOR, ARLINGTON, VA, 22202, US

(21) Appl. No.: 11/662,091

(22) PCT Filed: Sep. 13, 2005
(86) PCT No.: PCT/FR05/02264

§ 371 Date: Mar. 7, 2007

(30) Foreign Application Priority Data
Sep. 17, 2004 (FR) 0409836

(51) Int. Cl. A61F 9/007

(52) U.S. Cl. 606/166

(57) ABSTRACT

Device for cutting a corneal epithelial cover includes a fixing ring (1), a suction source (4), a cutting head (5) which is movably mounted on the ring (1) and an oscillating blade (10), elements (7, 8) for actuating the cutting head movements with respect to the ring and elements for controlling a suction, the blade oscillation and actuation adjustment according to: a starting phase of the corneal cover cutting, wherein the suction level is maximum, the advancing speed of the head is minimum and the oscillation frequency of the blade is maximum, a phase for cutting the larger part of the corneal cover, wherein the advancing speed is maintained or increased and the oscillation speed and the suction level are maintained or reduced and a third phase, wherein the head speed is inverted, oscillations are stopped and the suction level is maintained at the reduced level of the second phase.

Refractive surgery is applied to the cornea of the eye so as to modify its shape for the purpose of correcting or improving its optical properties.

### BACKGROUND OF THE INVENTION

In this field, surgical procedures are constantly changing, since they are continuously taking account of information, summaries, conclusions, and hypotheses that result from operations that have been performed and followed up over several tens of years.

At present, some of the experimentation is directed towards implementing an operation on the cornea that consists in removing an epithelial flap, in acting on the surface uncovered thereby, and in replacing the epithelial flap on the zone in question as well as possible, given the quality of the epithelial flap.

One of the difficulties in that procedure lies in making an epithelial flap of acceptable quality that can be folded back down onto the field where action has been taken.

### OBJECTS AND SUMMARY OF THE INVENTION

An object of the invention is to give surgeons means for performing such cutting of the epithelial flap under the best possible conditions.

To this end, the invention thus provides an apparatus for cutting an epithelial corneal flap, the apparatus comprising:
a fastener ring that can be held stationary on the eye of the patient by suction;
a suction source connected to the ring, and capable of creating a level of suction in the ring that is adjustable;
a cutter head mounted to move on the ring and guided relative thereto in a scan plane, the cutter head comprising:
   a blade with an active edge that is dull and rectilinear and members for guiding movement of the blade relative to the cutter head parallel to the above-mentioned active head;
   drive means for driving the blade to perform oscillating motion that is adjustable in frequency relative to the cutter head; and
actuator means for driving the movement of the cutter head relative to the ring at a speed that is adjustable;
the apparatus comprising control means for controlling said suction, drive, and actuator means so as to create a cutting cycle comprising:
a first stage of starting cutting of the corneal flap, in which stage the level of suction is at a maximum and the speed of advance of the cutter head is established before engagement at a minimum value, while the value at which the level of the frequency of oscillation of the blade is established is a maximum value;
a second stage of cutting the major fraction of the corneal flap, in which stage the speed of advance is maintained or increased, while the frequency of oscillation and the level of suction are decreased, or at most conserved; and
a third stage in which the speed of the head is reversed, the oscillations are stopped, and the level of suction is maintained at the diminished level of the second stage.

Experiment has shown that using a cutter blade that is dull makes it possible to split the cornea in lamellar manner along cleavage planes thereof, i.e. in planes where there is a change in the mechanical characteristics between the layers of the cornea. Thus, for example, the epithelial layer rests on a membrane referred to as Bowman's membrane, which has the characteristic of withstanding penetration better than the epithelial layer itself. Thus, by using a dull blade, it is possible to perform lamellar dissection immediately above Bowman's membrane. It should be understood that a blade that is said to be “dull”, is a blade having a cutting edge that is not very sharp because it has been sharpened in a special way, and that might include specific surface finishing. Nevertheless, the question of starting the cut remains to be solved. In known techniques, cutting is started by means of a sharp blade such as a trephine that is caused to penetrate into the cornea down to a depth that is substantially equal to the assumed thickness of the epithelial layer, and then the blade is changed so as to perform lamellar dissection using a dull blade. That procedure is complicated and somewhat random, leading to a lack of uniformity and of repeatability in surgery. By various adjustments of parameters such as the level of suction, the speed of advance, and the frequency of oscillation of the blade, the above-specified sequence of phases has been determined experimentally, and it makes it possible to proceed with a satisfactory start of cutting followed by cutting of the lamellar dissection type that is also satisfactory, while also determining conditions that make it possible to eliminate practically all risk of the blade penetrating through Bowman's membrane. Finally, the third stage is defined so that the return of the cutter blade takes place with the least possible chance of traumatizing the epithelial flap that has just been made.

Preferably, the above-mentioned control means include a pedal coupled to the above-mentioned suction, drive, and actuator means via programmable computer means. It is also advantageous for the times during which each of the stages are performed to be programmed while the beginnings of each of those stages are triggered by the user. The set of parameters may be entirely programmable by the user, or it may be entirely predetermined in the factory.

### BRIEF DESCRIPTION OF THE DRAWING

Other characteristics and advantages of the invention appear from the following description of an embodiment.

Reference is made to the accompanying drawing, in which:

FIG. 1 is a diagrammatic overall view of apparatus in accordance with the invention; and

FIG. 2 is a draft showing the three stages I, II, III in the operation of the apparatus in accordance with the invention.

### MORE DETAILED DESCRIPTION

The figures show the apparatus of the invention diagrammatically. In a manner that is conventional and similar to the microkeratomes used for refractive surgery known as “lasik”, the apparatus includes a fastener ring 1 for applying to the eyeball 2 and held thereon by means of suction created in the annular chamber 3 that is closed by the eyeball 2, the vacuum being established by a vacuum pump 4 having conventional means 12 for adjusting the level the suction, and thus the vacuum, established in the annular chamber 3.

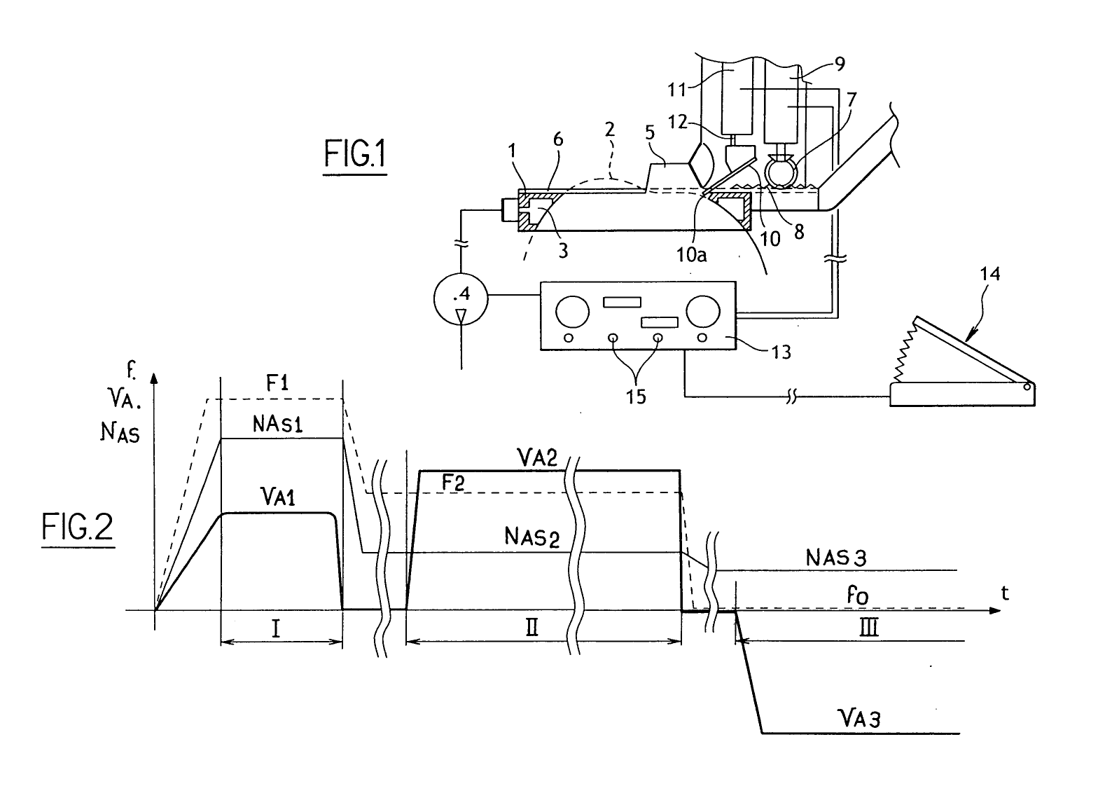

This ring defines a scanning plane for a cutter head 5 that is moved in said plane along a path that is rectilinear or circular under the control of guide means cooperating between the ring and the cutter head. In FIG. 1, the guide means are constituted by rectilinear slides 6, and the means for driving the head 5 along the slides 6 are represented diagrammatically by cooperation between a pinion 7 and a stationary rack 8. The pinion 7 is driven by a motor 9 at speed that is adjustable. Naturally numerous other mechanisms could be implemented to provide these movements.

Within the cutter head, the apparatus includes a cutter blade 10 mounted to slide in a housing that extends substantially transversally relative to the path of the cutter head 5, said cutter blade having an active edge 10a that is dull. It is driven to oscillate parallel to its active edge by means of a motor 11 and an eccentric 12. The frequency of oscillation depends on the speed of rotation of the eccentric 12, which frequency is adjustable by controlling the motor 11.

The apparatus includes an electronic console 13 for controlling the keratome, via which it is possible to determine operating setpoints for each of the motor members such as the pump 4, the motor 9, and the motor 11, the electronic control unit receiving as inputs signals coming from the surgeon, such as signals from a pedal 14 operated by the surgeon's foot, for example. The electronic unit 13 also has means 15 for displaying the above-mentioned setpoint values in order to determine an operating cycle dedicated to the patient for operation and determined by the surgeon who has examined the patient. The surgeon may have a variety of control pedals all connected to the electronic unit. For example, one pedal may be dedicated to triggering suction, another (with a variety of thresholds) to cutting, and a third to returning the cutter head.

When the operation is to be performed, the surgeon determines the various parameters, or the various operating setpoints for the apparatus. Thus, the operator selects a first frequency F1 for oscillation of the blade 10, a first level of suction such as NAS1 to be established in the ring, and a first speed of advance for the head along its path VA1. A first pulse or a first depression of the pedal 14 (or using a separate pedal) triggers performance of stage I as shown in FIG. 2. The cutter head is at the beginning of its path situated away from any contact with the cornea, so that there is time to reach the setpoint values before taking any action on the eye. The flap then begins to be cut with the variables F1, NAS1, and VA1, and this continues for a length of time that is either predetermined or else assessed by the surgeon who, on deciding that the starting operation is complete, can release the pedal. At that moment, the speed of advance of the head drops to zero while the frequency of oscillation of the blade decreases to reach a setpoint value F2, and likewise the level of suction NAS2 becomes established in the ring 3. It is possible for F2 and NAS2 to be maintained equal to F1 and NAS2, but in any event they are not increased.

The surgeon then causes a second pulse to be delivered, or depresses the pedal 14 a second time, and the cutting of the epithelial flap continues using the values F2, NAS2, and a speed of advance VA2 that is faster than the preceding speed VA1. In like manner, the end of this operation of cutting the main portion of the corneal flap occurs either in pre-programmed manner, or else at the initiative of the surgeon. At the end of this second stage, referenced II in FIG. 2, the frequency of oscillation drops to zero as does the speed of advance, while the level of suction remains held at its threshold NAS2, or is further reduced to NAS3.

A third pulse on the pedal 14, or operating a separate pedal, causes the travel direction of the cutter head 5 on the ring 1 to be reversed at a setpoint speed VA3 so as to be able to withdraw the cutter head after preparing the epithelial flap and putting into place the laser bombardment for modifying the surface uncovered by making this lamellar flap.

The surgeon terminates the operation by folding the flap back down onto the field that has been sculpted by the laser.

It should be observed that the above-mentioned programming of the various parameters of the procedure may include steps of sensing physical magnitudes, such as the power supply currents drawn by the motors, which magnitudes can be sensed continuously, and comparing them with rates that are predetermined for said magnitudes and representative of how a stage of the surgical procedure is to proceed.

1. An apparatus for cutting an epithelial corneal flap, the apparatus comprising:
a fastener ring (1) that can be held stationary on the eye (2) of the patient by suction;
a suction source (4) connected to the ring (1), and capable of creating a level of suction (NAS) in the ring that is adjustable;
a cutter head (5) mounted to move on the ring (1) and guided relative thereto in a scan plane, the cutter head comprising: a blade (10) with an active edge (10a) that is dull and rectilinear; members for guiding movement of the blade (10) relative to the cutter head parallel to the above-mentioned active head (10a); and drive means (12) for driving the blade (10) to perform oscillating motion that is adjustable in frequency relative to the cutter head (5); and
actuator means (7, 8) for driving the movement of the cutter head relative to the ring at a speed (VA) that is adjustable;
the apparatus including control means for controlling said suction, drive, and actuator means so as to create a cutting cycle comprising:
a first stage (I) of starting cutting of the corneal flap, in which stage the level of suction (NAS1) is at a maximum and the speed of advance (VA1) of the cutter head is established before engagement at a minimum value, while the value at which the level (F1) of the frequency of oscillation of the blade is established is a maximum value;
a second stage (II) of cutting the major fraction of the corneal flap, in which stage the speed of advance (VA2) is maintained or increased, while the frequency (F2) of oscillation and the level of suction (NAS2) are decreased, or at most conserved; and
a third stage (III) in which the speed (VA3) of the head (5) is reversed, the oscillations are stopped, and the level of suction is maintained at the diminished level of the second stage.

2. Apparatus according to claim 1, wherein the above-mentioned control means include a pedal (14) coupled to the above-mentioned suction, drive, and actuator means (4, 9, 11) via programmable computer means (15).

3. Apparatus according to claim 1, including means for programming the time over which each stage (I, II, III) takes place, with the beginning of each stage being triggered by the user.

4. Apparatus according to claim 2 means for programming the time over which each stage (I, II, III) takes place, with the beginning of each stage being triggered by the user.
